(19) United States
(12) Patent Application Publication
(10) Pub. No.: US 2007/0087441 A1
(43) Pub. Date: Apr. 19, 2007

(54) Impurity of anastrozole intermediate, and uses thereof

(76) Inventors: Alessandro Pontiroli, S. Maria della Versa (PV) (IT); Roberto Casalone, Santhia (VC) (IT)

Correspondence Address: KENYON & KENYON LLP, ONE BROADWAY, NEW YORK, NY, 10004, US

(21) Appl. No.: 11/476,396

(22) Filed: Jun. 27, 2006

Related U.S. Application Data
(60) Provisional application No. 60/694,528, filed on Jun. 27, 2005.

(51) Int. Cl. G01N 33/00; C07C 255/63

(52) U.S. Cl. 436/109; 558/419

(57) ABSTRACT

Reference markers and reference standards for the determination of impurities in Anastrozole are provided.

### RELATED APPLICATIONS

This application claims the benefit of U.S. Provisional Application No. 60/694,528, filed Jun. 27, 2005, and claims priority to U.S. Patent Application No. Awaited, filed Jun. 27, 2006, herein incorporated by reference.

### FIELD OF INVENTION

The present invention relates to an impurity of an Anastrozole intermediate, referred to as “impurity A” and uses thereof.

### BACKGROUND OF THE INVENTION

Anastrozole, of the chemical name 1,3-benzenediacetonitrile-α,α,α′,α′-tetramethyl-5-(1H-1,2,4-triazol-1-ylmethyl) and having the following chemical structure, 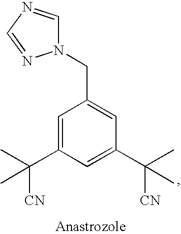
is a potent and selective non-steroidal inhibitor of the aromatase (oestrogen synthetase) system, which converts adrenal androgens to oestrogens in peripheral tissue. It is used in the treatment of advanced or locally advanced breast cancer, and as adjuvant treatment in early breast cancer, in postmenopausal women. This drug is available commercially for oral administration ARIMIDEX® by AstraZeneca.

Like any synthetic compound, Anastrozole can contain extraneous compounds or impurities that can come from many sources. They can be unreacted starting materials, by-products of the reaction, products of side reactions, or degradation products. Impurities in Anastrozole or any active pharmaceutical ingredient (API) are undesirable and, in extreme cases, might even be harmful to a patient being treated with a dosage form containing the API.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during processing of an API, such as (S)-anastrozole, it must be analyzed for purity, typically, by HPLC or TLC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. As discussed above, in the United States, the Food and Drug Administration guidelines recommend that the amounts of some impurities be limited to less than 0.1 percent.

Generally, side products, by-products, such as the impurity A, and adjunct reagents (collectively “impurities”) are identified spectroscopically and/or with another physical method, and then associated with a peak position, such as that in a chromatogram, or a spot on a TLC plate. (Strobel p. 953, Strobel, H. A.; Heineman, W. R., Chemical Instrumentation: A Systematic Approach, 3rd dd. (Wiley & Sons: New York 1989)). Thereafter, the impurity can be identified, e.g., by its relative position on the TLC plate and, wherein the position on the plate is measured in cm from the base line of the plate or by its relative position in the chromatogram of the HPLC, where the position in a chromatogram is conventionally measured in minutes between injection of the sample on the column and elution of the particular component through the detector. The relative position in the chromatogram is known as the “retention time.”

The retention time can vary about a mean value based upon the condition of the instrumentation, as well as many other factors. To mitigate the effects such variations have upon accurate identification of an impurity, practitioners use the “relative retention time” (“RRT”) to identify impurities. (Strobel p. 922). The RRT of an impurity is its retention time divided by the retention time of a reference marker. It may be advantageous to select a compound other than the API that is added to, or present in, the mixture in an amount sufficiently large to be detectable and sufficiently low as not to saturate the column, and to use that compound as the reference marker for determination of the RRT.

Those skilled in the art of drug manufacturing research and development understand that a compound in a relatively pure state can be used as a “reference standard.” A reference standard is similar to a reference marker, which is used for qualitative analysis only, but is used to quantify the amount of the compound of the reference standard in an unknown mixture, as well. A reference standard is an “external standard,” when a solution of a known concentration of the reference standard and an unknown mixture are analyzed using the same technique. (Strobel p. 924, Snyder p. 549, Snyder, L. R.; Kirkland, J. J. Introduction to Modern Liquid Chromatography, 2nd ed. (John Wiley & Sons: New York 1979)). The amount of the compound in the mixture can be determined by comparing the magnitude of the detector response. See also U.S. Pat. No. 6,333,198, incorporated herein by reference.

The reference standard can also be used to quantify the amount of another compound in the mixture if a “response factor,” which compensates for differences in the sensitivity of the detector to the two compounds, has been predetermined. (Strobel p. 894). For this purpose, the reference standard is added directly to the mixture, and is known as an “internal standard.” (Strobel p. 925, Snyder p. 552).

The reference standard can serve as an internal standard when, without the deliberate addition of the reference standard, an unknown mixture contains a detectable amount of the reference standard compound using the technique known as “standard addition.”

In the “standard addition technique”, at least two samples are prepared by adding known and differing amounts of the internal standard. (Strobel pp. 391-393, Snyder pp. 571, 572). The proportion of the detector response due to the reference standard present in the mixture without the addition can be determined by plotting the detector response against the amount of the reference standard added to each of the samples, and extrapolating the plot to zero concentration of the reference standard. (See, e.g., Strobel, FIG. 11.4 p. 392). The response of a detector in HPLC (e.g. UV detectors or refractive index detectors) can be and typically is different for each compound eluting from the HPLC column. Response factors, as known, account for this difference in the response signal of the detector to different compounds eluting from the column.

As is known by those skilled in the art, the management of process impurities is greatly enhanced by understanding their chemical structures and synthetic pathways, and by identifying the parameters that influence the amount of impurities in the final product.

The detection or quantification of the reference standard serves to establish the level of purity of the API or intermediates thereof. Use of a compound as a standard requires recourse to a sample of substantially pure compound.

### SUMMARY OF THE INVENTION

In one aspect, the present invention provides a newly isolated impurity A, 2,3-‘Bis-[3-(cyano-dimethyl-methyl)-5-methyl-phenyl]-2-methyl-propionitrile of the following formula.

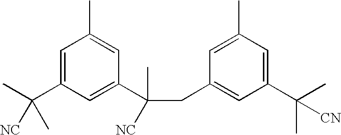

In another aspect, the present invention provides a process of determining the presence of a compound in a sample comprising carrying out HPLC or TLC with an impurity A as a reference marker.

In yet another aspect, the present invention provides a process of determining the presence of impurity A in a sample comprising carrying out HPLC or TLC with the impurity A as a reference marker. Specifically, this process comprises:

(a) determining by HPLC or TLC the retention time corresponding to impurity A in a reference marker comprising the impurity A;

(b) determining by HPLC or TLC the retention time corresponding to impurity A in a sample comprising 3,5-bis(2-cyanoisopropyl)toluene and impurity A; and

(c) determining the presence of impurity A in the sample by comparing the retention time of step (a) to the retention time of step (b).

In one aspect, the present invention provides a process of determining the amount of a compound in a sample comprising carrying out HPLC or TLC with an impurity A as a reference standard.

In another aspect, the present invention also provides a process for preparing anastrozole from 3,5-bis(2-cyanoisopropyl)toluene having less than about 0.10% area by HPLC of impurity A is present which comprises the steps of:

(a) obtaining one or more samples of one or more batches of 3,5-bis(2-cyanoisopropyl)toluene;

(b) measuring the level of impurity A in each of the samples;

(c) selecting a batch from step a) having a level of impurity A of about less than 0.10 area % by HPLC, based on the measurement of the samples from the batches; and

(d) using the selected batch to prepare anastrozole.

In yet another aspect, the present invention provides an HPLC method used to determine the presence of impurity A in a 5-bis(2-cyanoisopropyl)toluene sample comprising: combining a 5-bis(2-cyanoisopropyl)toluene sample with water to obtain a solution; injecting the obtained solution into a 100 mm×4.6 mm mm HYPERSIL BDS C18 (or similar) column; eluting the sample from the column at about 35 minutes using water (referred herein as “eluent A”) and acetonitrile (referred to herein as “eluent B”) as an eluent, and measuring the impurity A content in the relevant sample with a UV detector (preferably at a 210 nm wavelength).

In one embodiment, the present invention provides pharmaceutical composition comprising Anastrozole made by the process of the invention and pharmaceutically acceptable excipients.

In another embodiment, the present invention provides a process for preparing pharmaceutical formulation comprising mixing Anastrozole made by the process of the invention and a pharmaceutically acceptable carrier.

### DETAILED DESCRIPTION OF THE INVENTION

The term “substantially pure” in reference to 3,5-bis(2-cyanoisopropyl)toluene refers to 3,5-bis(2-cyanoisopropyl)toluene containing less than about 0.10% area by HPLC of impurity A.

The term “substantially pure” in reference to Anastrozole refers to Anastrozole containing less than about 0.10% area by HPLC of impurity B, as defined below.

The present invention provides a newly isolated impurity, 2,3-Bis-[3-(cyano-dimethyl-methyl)-5-methyl-phenyl]-2-methyl-propionitrile of the following formula.

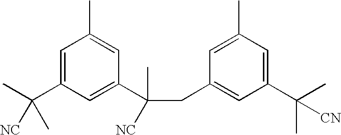

This impurity, referred to as “impurity A”, contaminates an Anastrozole intermediate, 3,5-bis(2-cyanoisopropyl)toluene of formula I.

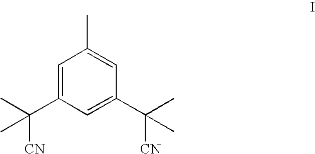

It can be characterized by data selected from the group consisting of an RRT at about 1.53 in relation to 3,5-bis(2-cyanoisopropyl)toluene of formula I and/or an M/S spectra with an m/z peak at about 406.

Impurity A may be isolated by column chromatography using a mixture of heptane and ethylacetate as an eluent. Preferably, the eluent contains with heptane and ethylacetate in a ratio of about 9:1, respectively. Preferably, impurity A contains about 0% to about 10% area by HPLC of 3,5-bis(2-cyanoisopropyl)toluene of formula I.

It was found by the inventors of the present invention that impurity A converts during the course of the reaction for preparing Anastrozole from 3,5-bis(2-cyanoisopropyl)toluene of formula I to an impurity that contaminates Anastrozole, referred to as “impurity B”, of the following structure, 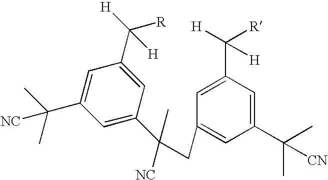
wherein R and R′ can be independently, H or 1,2,4-triazole. The conversion of impurity A to impurity B is illustrated by the following scheme:

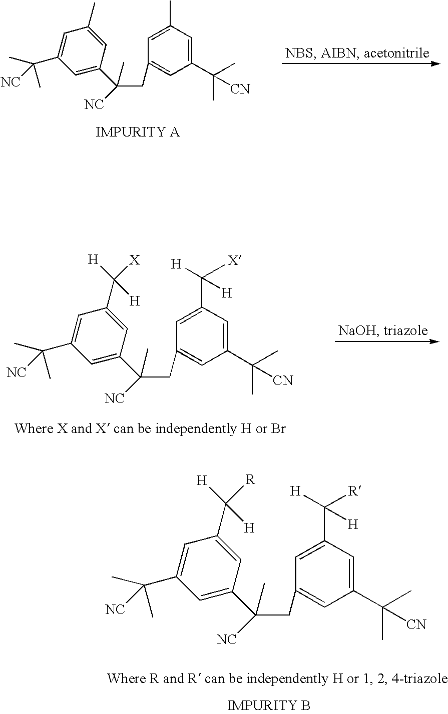

The conversion is in such a way that the amount of impurity A is very similar to the amount of impurity B. Moreover, since this impurity is characterized by a similar solubility to Anastrozole, it is difficult to separate it from Anastrozole, and hence, to use it as a reference marker and standard. Thus, combining the above knowledge with the fact that the inventors of the present invention found that impurity A can be separated more easily and efficiently from the starting material, 3,5-bis(2-cyanoisopropyl)toluene of formula I, makes its use as a reference marker and a reference standard more attractive.

The present invention further provides a process of determining the presence of a compound in a sample comprising carrying out HPLC or TLC with impurity A as a reference marker.

The present invention also provides a process of determining the presence of impurity A in a sample comprising carrying out HPLC or TLC with the impurity A as a reference marker. Specifically, this process comprises:

(a) determining by HPLC or TLC the retention time corresponding to impurity A in a reference marker comprising the impurity A;

(b) determining by HPLC or TLC the retention time corresponding to impurity A in a sample comprising 3,5-bis(2-cyanoisopropyl)toluene and impurity A; and

(c) determining the presence of impurity A in the sample by comparing the retention time of step (a) to the retention time of step (b).

The present invention provides a process of determining the amount of a compound in a sample comprising carrying out HPLC or TLC with an impurity A as a reference standard.

The present invention further provides a method of quantifying the amount of impurity A in a sample comprising performing a HPLC or TLC, wherein the impurity αis used as a reference standard. Specifically, this process comprises the steps of:

(a) measuring by HPLC or TLC, the area under a peak corresponding to impurity A in a reference standard comprising a known amount of impurity A;

(b) measuring by HPLC or TLC, the area under a peak corresponding to impurity A in a sample comprising impurity A and 3,5-bis(2-cyanoisopropyl)toluene; and

(c) determining the amount of impurity A, in the sample by comparing the area of step (a) to the area of step (b).

The present invention also provides a process for preparing anastrozole from 3,5-bis(2-cyanoisopropyl)toluene having less than about 0.10% area by HPLC of impurity A is present which comprises the steps of:

(a) obtaining one or more samples of one or more batches of 3,5-bis(2-cyanoisopropyl)toluene;

(b) measuring the level of impurity A in each of the samples;

(c) selecting a batch from step a) having a level of impurity A of about less than 0.10 area % by HPLC, based on the measurement of the samples from the batches; and

(d) using the selected batch to prepare anastrozole.

In a case wherein the level measured in step b) is higher than about 0.10 area % by HPLC, the process further comprises the step of purification by any means known in the art, including the method disclosed in the U.S. Provisional Patent Application No. 60/694,528, wherein 3,5-bis(2-cyanoisopropyl)toluene is crystallized from a solvent selected from the group consisting of C₆₋₉ aromatic hydrocarbons and C₂₋₈ ethers.

The present invention provides an HPLC method used to determine the presence and amount of impurity A in a 3,5-bis(2-cyanoisopropyl)toluene sample comprising: combining a 5-bis(2-cyanoisopropyl)toluene sample with water to obtain a solution; injecting the obtained solution into a 100 mm×4.6 mm mm HYPERSIL BDS C18 (or similar) column; eluting the sample from the column at about 35 minutes using water (referred herein as “eluent A”) and acetonitrile (referred to herein as “eluent B”) as an eluent, and measuring the impurity A content in the relevant sample with a UV detector (preferably at a 210 nm wavelength).

Preferably, the eluent used may be a mixture of eluent A and eluent B, wherein the ratio of them varies over the time, i.e. a gradient eluent. At the time 0 minutes, the eluent contains 80% of eluent A and 20% of eluent B. At 30 minutes, the eluent contains 40% of eluent A and 60% of eluent B. At 35 minutes, the eluent contains 20% of eluent A and 80% of eluent B, while at 36 minutes; the eluent contains 80% of eluent A and 20% of eluent B.

The present invention further provides pharmaceutical composition comprising Anastrozole made by the process of the invention and pharmaceutically acceptable excipients.

The present invention also provides a process for preparing pharmaceutical formulation comprising mixing Anastrozole made by the process of the invention and a pharmaceutically acceptable carrier.

Having described the invention with reference to certain preferred embodiments, other embodiments will become apparent to one skilled in the art from consideration of the specification. The invention is further defined by reference to the following examples describing in detail the preparation of the compound of the present invention. It will be apparent to those skilled in the art that many modifications, both to materials and methods, may be practiced without departing from the scope of the invention.

### EXAMPLES

The Analysis of Impurity a is Done in 3,5-bis(2-cyanoisopropyl)toluene Crude Using the Following HPLC:

| Column & Packing: | HYPERSIL BDS C18; 3 μm, 100 mm × | | |
|---|---|---|---|
| | 4.6 mm, cat n. 28103-104630 or equivalent | | |
| Eluent A: | Water | | |
| Eluent B: | Acetonitrile | | |
| | Time | % Eluent | % Eluent |
| | (min) | A | B |
| Gradient | 0 | 80 | 20 |
| | 30 | 40 | 60 |
| | 35 | 20 | 80 |
| | 36 | 80 | 20 |

| | |
|---|---|
| Stop time: | 35 minutes |
| Equilibrium time: | 5 minutes |
| Flow Rate: | 1.0 ml/min. |
| Detector: | UV at 210 nm |
| Column temperature: | 60° C. |
| Injection: | 5 μl |
| Diluent: | Acetonitrile |

The Mobile phase composition and flow rate may be varied in order to achieve the required system suitability.
Mass-Spectrum Analysis:

Direct infusion into ESI ion source. The operative conditions employed were the following:

| | |
|---|---|
| Instrument: | LCQ Deca (Thermofinnigan), operating in |
| | positive ion mode |
| Sample concentration: | 10⁻⁶ M in Acetonitrile |
| Spray Voltage: | 4 kV |
| Capillary Voltage: | 13 V |
| Capillary temperature: | 270° C. |

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention. The examples set forth below describe single crystallization experiments, which can be repeated to obtain the same yields and improvements in purification until the final desired purity is obtained.

### Example 1

### Crystallization of 3,5-bis(2-cyanoisopropyl)toluene From Toluene

A 4 g sample of 3,5-bis(2-cyanoisopropyl)toluene, having an initial impurity A content of 1.93 HPLC area percent, was suspended in 10 ml of toluene, and heated to 65° C., until complete dissolution occurs. The solution was then allowed to cool to 25° C. over a period of 1 hour, and then cooled to 0° C. over a period of 2 hours. After 30 min at 0° C., the resulting suspension was filtered, and the filtrate is rinsed with 2.5 ml of toluene, pre-cooled to 0° C. Purified 3,5-bis(2-cyanoisopropyl)toluene was recovered in an amount of 3.2 g, having an impurity A content of 1.02 HPLC area percent.

### Example 3

### Crystallization and Recrystallization of 3,5-bis(2-cyanoisopropyl)toluene From Toluene

3,5-bis(2-cyanoisopropyl)toluene (50 g), containing 0.45% of impurity A, was dissolved in toluene (150 ml) and heated to 65-70° C. until a complete solution was obtained. After 10 minutes, the solution was then allowed to cool to 25° C. in 6 hours. After this time, the suspension was cooled to −20° C. in 1 hour, stirred at the same temperature for 30 minutes and then filtered. The solid was then washed with toluene (25 ml) pre-cooled to −20° C.

The wet solid was then analyzed via HPLC showing a content of 0.24% of impurity A. Recrystallizing this solid two more times gave 3,5-bis(2-cyanoisopropyl)toluene having 0.07% of impurity A. this solid was then dried in oven at 50° C. until all solvent were removed.

### Example 4

### Crystallization of 3,5-bis(2-cyanoisopropyl)toluene from 3 Volumes of Toluene

A 42 g sample of 3,5-bis(2-cyanoisopropyl)toluene, having an initial impurity A content of 0.11 HPLC area percent was suspended in 130 ml of toluene, and heated to 61° C., until complete dissolution occurred. The solution was then allowed to cool to 25° C. over a period of 3 hours obtaining a suspension, and then cooled to −20° C. over a period of 2 hours. After 30 min at −20° C., the resulting suspension was filtered, and the filtrate was rinsed with 2.5 ml of toluene that was pre-cooled to −20° C. Purified 3,5-bis(2-cyanoisopropyl)toluene was recovered in an amount of 40.1 g, having an impurity A content of 0.06 HPLC area percent.

### Example 4

### Synthesis of Anastrozole

A 30 g sample of the 3,5-bis(2-cyanoisopropyl)toluene having 0.06% area by HPLC of impurity A, was dissolved in 150 ml of acetonitrile, and 24.8 g of N-bromosuccinimide were added. The resulting suspension was heated to 50° C. for 30 minutes, until a light yellow solution was obtained. Then, 0.5 g of 2,2′-azobis(2-methylpropionitrile) was added, and the reaction was heated to 70° C. for 6 hours. The solution was then allowed to cool to 20° C., and poured into 150 ml of a 5 percent by weight solution of sodium metabisulphite in water with vigorous stirring. The organic layer was then separated and washed with 100 ml of a 5 percent by weight solution of sodium carbonate in water before removing the organic solvent under reduced pressure, until a total volume of 90 ml was obtained. The resulting slurry was then heated to 50° C., and 150 ml of heptane were slowly added over a period of 30 minutes, raising the temperature to 70° C. The suspension was then allowed to cool to 20° C., and filtered on a sintered glass funnel. Drying under reduced pressure yields 54 g of crude 1-bromo-3,5-bis(2-cyanoisopropyl)toluene in 85 percent purity (HPLC).

B: Formation of Anastrozole:

A 16.7 g sample of 1,2,4-triazole was dissolved in 52 ml of NMP at 20° C., and 9.7 g of NaOH was added in portions over 1 hour, while maintaining the temperature at less than 35° C. The solution was stirred for 18 hours at 20° C., and then cooled to −30° C. A solution of 40 g of crude alpha-bromo-3,5-bis(2-cyanoisopropyl)toluene in 60 ml of NMP was slowly added over 6 hours, while maintaining the temperature below −20° C. At the end of the addition, the suspension was stirred for 18 hours at −20° C., and, during that time, the reaction was monitored via HPLC. When the amount of starting material was less than 0.5 percent, acetic acid was added in an amount sufficient to provide a pH of about 6.5 to about 7. The mixture was slowly allowed to warm to 20° C., then 120 ml of toluene, 240 of heptane, and 170 ml of water were added. The biphasic system was stirred vigorously for 30 minutes, and the organic layer was then separated. Then, 240 ml of water, 60 ml of toluene, and 120 ml of heptane were added to the aqueous phase, and the system was stirred for 30 minutes before the organic phase was separated. Then, 400 ml of toluene and 240 ml of water were added to the aqueous portion, and the biphasic system was stirred for 1 hour. The organic layer was separated, and washed 3 times with 180 ml of a 0.05N solution of sulphuric acid in water. The final organic phase was concentrated under reduced pressure to a final volume of 150 ml at 40° C., and 180 ml of heptane were added drop-wise over a period of 1 hour. The suspension was cooled to 0° C., stirred for 1 hour, and filtered. The crude solid was dissolved in 390 ml of 2-propanol at 50° C., and 78 ml of heptane were slowly added under stirring.

The solution was cooled to 0° C., stirred for 1 hour, and filtered. The solid was dried at 55° C. under reduced pressure until a constant weight was achieved; producing 23.5 g of product with a purity of greater than 99.4 HPLC area percent having 0.06% of impurity B, and a melting point of 85° C., as measured by DSC.

### Example 5

### Isolation of Impurity A

A sample of 3,5-bis(2-cyanopropyl)toluene containing impurity A was purified by flash column chromatography eluting with a 9:1 mixture of heptane/ethyl acetate and analyzing the fractions with HPLC. Fractions containing impurity A with purity >90% were pooled and the solvent was removed under vacuum to afford impurity A (2,3-Bis-[3-(cyano-dimethyl-methyl)-5-methyl-phenyl]-2-methyl-propionitrile) of formula I.

What is claimed:

1. Isolated 2,3-Bis-[3-(cyano-dimethyl-methyl)-5-methyl-phenyl]-2-methyl-propionitrile (impurity A), of the following formula:

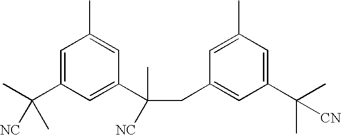

2. The isolated impurity of claim 1, containing about 0% to about 10% of 3,5-bis(2-cyanoisopropyl)toluene.

3. The isolated impurity of claim 2, wherein it contaminates an Anastrozole intermediate, 3,5-bis(2-cyanoisopropyl)toluene of formula I:

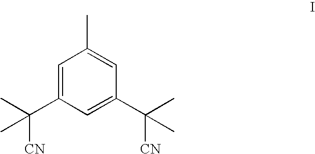

4. The isolated impurity of claim 2, characterized by an RRT at about 1.53 in relation to 3,5-bis(2-cyanoisopropyl)toluene of formula I.

5. The isolated impurity of claim 2, characterized by an M/S spectra with an m/z peak at about 406.

6. A process of determining the presence of a compound in a sample comprising carrying out HPLC or TLC with impurity A as a reference marker.

7. The process of claim 6, wherein the process is for determining the presence of impurity A in a sample comprising carrying out HPLC or TLC with the impurity A as a reference marker.

8. The process of claim 6, comprising:
(a) determining by HPLC or TLC the retention time corresponding to impurity A in a reference marker comprising the impurity A;
(b) determining by HPLC or TLC the retention time corresponding to impurity A in a sample comprising 3,5-bis(2-cyanoisopropyl)toluene and impurity A; and
(c) determining the presence of impurity A in the sample by comparing the retention time of step (a) to the retention time of step (b).

9. A process of determining the amount of a compound in a sample comprising carrying out HPLC or TLC with an impurity A as a reference standard.

10. The process of claim 6, wherein the process is for quantifying the amount of impurity A in a sample comprising performing a HPLC or TLC, wherein the impurity A is used as a reference standard.

11. The process of claim 6, comprising:
(a) measuring by HPLC or TLC, the area under a peak corresponding to impurity A in a reference standard comprising a known amount of impurity A;
(b) measuring by HPLC or TLC, the area under a peak corresponding to impurity A in a sample comprising impurity A and 3,5-bis(2-cyanoisopropyl)toluene; and
(c) determining the amount of impurity A, in the sample by comparing the area of step (a) to the area of step (b).

12. A process for preparing anastrozole from 3,5-bis(2-cyanoisopropyl)toluene having less than about 0.10% area by HPLC of impurity A comprising the steps of:
(a) obtaining one or more samples of one or more batches of 3,5-bis(2-cyanoisopropyl)toluene;
(b) measuring the level of impurity A in each of the samples;
(c) selecting a batch from step a) having a level of impurity A of about less than 0.10 area % by HPLC, based on the measurement of the samples from the batches; and
(d) using the selected batch to prepare anastrozole.

13. The process of claim 12, wherein the process further comprises, prior to step d, purification of 3,5-bis(2-cyanoisopropyl)toluene when the level measured in step b) is higher than about 0.10 area % by HPLC.

14. The process of claim 12, wherein the purification is done by crystallization of 3,5-bis(2-cyanoisopropyl)toluene from a solvent selected from the group consisting of C₆₋₉ aromatic hydrocarbon, and C₂₋₈ ether.

15. An HPLC method used to determine the presence and amount of impurity A in a 3,5-bis(2-cyanoisopropyl)toluene sample comprising:
(a) combining a 5-bis(2-cyanoisopropyl)toluene sample with water to obtain a solution;
(b) injecting the obtained solution into a column;
(c) eluting the sample from the column at about 35 minutes using water, eluent A, and acetonitrile, eluent B, as an eluent, and
(d) measuring the impurity A content in the relevant sample with a UV detector.

16. The method of claim 15, wherein the UV detector operates at wavelength of about 210 nm.

17. The process of claim 15, wherein the eluent used is a mixture of eluent A and eluent B.

18. The method of the claim 15, wherein the ratio between eluent A and eluent B varies over the time.

19. The method of claim of the claim 15, wherein at the time 0 minutes, the eluent contains 80% of eluent A and 20% of eluent B; at 30 minutes, the eluent contains 40% of eluent A and 60% of eluent B; at 35 minutes, the eluent contains 20% of eluent A and 80% of eluent B; and, at 36 minutes; the eluent contains 80% of eluent A and 20% of eluent B.

20. A pharmaceutical composition comprising Anastrozole made according to the process in claim 12 and pharmaceutically acceptable excipients.

21. A process for preparing pharmaceutical formulation comprising mixing Anastrozole made according to the process in claim 12 and a pharmaceutically acceptable carrier.
